(19) United States
(12) Patent Application Publication
(10) Pub. No.: US 2023/0015070 A1
(43) Pub. Date: Jan. 19, 2023

(54) HAND ANTI-STRESS AND/OR REHABILITATION DEVICE

(71) Applicant: Walter Alfredo RUIZ ARTAZO, San Juan (AR)

(72) Inventor: Walter Alfredo RUIZ ARTAZO, San Juan (AR)

(21) Appl. No.: 17/835,484

(22) Filed: Jun. 8, 2022

(30) Foreign Application Priority Data
Jul. 14, 2021 (ES) 202131472U

(51) Int. Cl. A61H 1/02

(52) U.S. Cl. CPC A61H 1/0285; A61H 2201/1635

(57) ABSTRACT

A hand anti-stress and or rehabilitation device that has two support bases where a metal rod stands with rounded rotating parts incorporating magnets that are fixed, or have a mobile union that allows to exert pressure on them, is disclosed. Optionally, it has, coupled fixed or dismountable, at each of the rod ends, a spring, or similar, at the distal end thereof, a lateral gripping part with four circular recesses to insert the fingers.

### OBJECT OF THE INVENTION

The invention, as stated in the title of this specification, refers to a hand anti-stress and/or rehabilitation device that contributes, to the function to which it is designed, with advantages and characteristics that are disclosed in detail thereafter.

The object of this invention refers to a device the structure of which, designed to be placed on a table and comprising the inclusion of one or more bodies provided with magnets that rotate about an axis when the hands passes on them, is specifically designed as a relaxing means to channel the tension and the stress, for example, at the office, because it helps to massage the hands nerve endings, serving as a tool of magnetotherapy and that likewise can serve as a means to exercise and rehabilitate the hand and adjacent areas, such as the wrist and the forearm and to prevent diseases generated by the work at the office and/or similar activities as it stretches out the muscles of the hand and/or the wrist.

### FIELD OF APPLICATION OF THE INVENTION

The field of application of this invention is within the sector of the industry engaged in manufacturing devices, tools and jigs for channelling the stress and to do manual exercises.

### BACKGROUND OF THE INVENTION

As it is known, in the market, different types and models of jigs and devices exists designed to exercise the hands to relieve the stress and/or to strengthen and move the muscles of the hands. Normally, they are apparatuses having a mechanical action that:
are either formed by an elastic body that is pressed against the hand, for example ball-, sphere-, egg- or net-shaped,
or that include parts attached by springs to press with the hand or with the fingers,
or that are flexible rod-shaped formed by a spring with handles, to be pressed with both hands.

Devices or jigs are likewise known having a relaxing or anti-stress action, normally formed by mobile elements to be moved, touched and/or pressed, for example a cube, dice-shaped with buttons for pressing, or for example sphere-shaped toys, etc.

On the other hand, the action that the magnetism exerts on the body is known. In fact, the magnetotherapy is a therapeutic treatment based on applying magnetic fields, usually used by physiotherapists and rehabilitation centers.

In turn, the magnetic therapy, magnetotherapy or biomagnetism is a practice of alternative medicine that implies the use of static magnetic fields produced by permanent magnets that possesses beneficial effects for the health.

Well, the objective of this invention is to provide the market with a new device matching, in a same element:
anti-stress means, to provide the user relaxation, namely in the professional field, for users who work in offices during long working days and that almost always do it using the hands to typing on the computer and/or holding the mouse, to be able to relieve tensions by channeling of the nerve ends with the effect of magnets, and
massage and rehabilitation means, to activate the mobility and strengthen the muscles of the hand as a whole, as well as those of the fingers and even of the wrist and forearm.

On the other hand, and as reference of the current state-of-the-art, although, as it was said, other devices are known, at least the applicant is not aware of the existence of any that matches both things in a single device, also of not any having technical and structural characteristics equal or similar to those that this device concretely shows, that are claimed herein.

### EXPLANATION OF THE INVENTION

The hand anti-stress and/or rehabilitation device that the invention proposes is configured as the suitable solution to the above-mentioned objective, the characterizing details that make it possible and that conveniently distinguishes it, appear in the final claims attached to this specification.

Concretely, what the invention proposes, as said before, is a device the structure of which with rotating elements provided with magnets is specifically designed as a relaxing means to channel the tension and the stress generated by the use of a computer or other manual elements, for example in the office, because it helps to massage the nerves ends of the hands, serving at same time as tool of magnetotherapy and that, in addition, optionally, it can also be used for the hand exercise and rehabilitation as well as for preventing muscle diseases, as well of the hand and fingers as of the wrist or forearm.

For all this, and more specifically, the device essentially comprises, two support bases between which a metal rod stands in which are inserted, with possibility of rotation on itself like an axis, one or more rounded parts, preferably three sphere-shaped parts, in which, in turn, magnets are radially incorporated distributed on its surface.

This way, for a preferred use of the device, the user takes with the index, middle, ring and little fingers the bases of the article with both hands and, with the thumbs, he rotates the spheres with magnets skewered in the central rod.

In addition, in the preferred embodiment, the device likewise comprises, coupled in the respective ends of the rod that acts as axis for the rotating bodies with the magnets, a spring or similar flexible threadlike element and at the distal end thereof, a lateral gripping part, preferably fitted with four circular recesses adapted to place the fingers (index, middle, ring and little fingers) inserted in them.

With this, for a preferred use of the device, the user, after resting the device on a table or similar flat surface, must place his fingers in the circles of the two gripping parts of the ends, can do movements in any direction the flexibility of the springs allows him. This permits to put pressure on the muscles of the hand and extend them. Also, he can do lateral, up and down movements (it also allows him to work the muscles of the writ and some of the forearm) and last, the possibility to contract more than one finger at a time.

Likewise, he can also do movements with the palm of the hand on rotating spheres with the magnets, provoking a relaxing effect on the nerves ends of the palm.

It must be said at this point that, although preferably the magnets are incorporated integral with the surface of the rotating parts, optionally one, several or all the magnets are incorporated by means of a mobile, rotating or flexible union, for example a strip, allowing that the magnets can be pressed with the hand or fingers and/or to rotate the magnets with respect to the spheres and, therefore, it offers thus a new relaxing and/or exercising functionality of the muscles.

On the other hand, it shall be pointed out that, in a preferred embodiment, the springs with the lateral gripping part with recesses for the fingers are dismountable elements in order that they can be withdrawn and used independently from the rest of the device, allowing to carry out with each of them exercises of pressure against the palm of the hand, which can serve to activate the muscles of the hand and forehand, strengthening for preventing injuries. In turn, the said dismountable nature allows that the device, that means, the rod with the rotating parts with magnets standing between the support bases, can be used without the lateral grips to do exercises of relaxation and magnetotherapy by passing softly the hand on them.

As for the support bases, that optionally are also dismountable elements, allowing that the device occupies less space for its storage and transport, in a preferred embodiment, are formed, each, by two hinged parts that allow to enlarge the lower support surface, in which, in turn, in a preferred embodiment, the incorporation of non-slip material has been provided.

Last, it shall be pointed out that, preferably, the lateral gripping parts with recesses for the fingers are of silicone, plastic, rubber or other similar soft material, to prevent that they can hurt, while the rest of parts, preferably, will be metallic although it is not discarded that they are also of plastic material.

### DESCRIPTION OF THE DRAWINGS

To complement the description that is being done and in order to assist to best understand the characteristics of the invention, attached to this specification, as an integral part thereof, there is a drawing in which, for illustration and not limitation purpose the following has been represented:

FIG. 1.—It shows a schematic top plan view of an example of embodiment of the hand anti-stress and/or rehabilitation device object of this invention, the parts and elements it comprises been seen, as well as their arrangement;

FIG. 2.—It shows a lateral elevation view of the example of the hand anti-stress and rehabilitation device of the invention shown in the FIG. 1;

FIG. 3.—It shows a top perspective view of the example of the device according to the invention appearing in the preceding figures, in this case including arrows indicating the different movements some of the parts presents;

FIGS. 4, 5 and 6.—They show another example of the device according to the invention in the same views as the FIGS. 1, 2 and 3, in this case an example that does not comprise any spring nor lateral gripping parts;

FIG. 7.—It shows a view in perspective of another example of the device of the invention, in this case an example with double support bases, mobile magnets and withdrawable lateral gripping parts.

FIGS. 8-A and 8-B.—They shown each views in perspective of one of the rotating parts of the device, according to an example of the FIG. 7, with mobile magnets, represented respectively in raised position and in retracted position; and

FIG. 9.—It shows a view in perspective of one of the withdrawable grips of the example of the device shown in the FIG. 7, representing independently the rest of the device.

### PREFERRED EMBODIMENT OF THE INVENTION

Seen the said figures and according to the numerals adopted, in them several non-limiting examples of embodiment of the hand anti-stress and/or rehabilitation device of the invention can be seen, which comprises what is described in detail below.

Thus, as it is observed in the said figures, the device (1) of the invention comprises, basically, two support bases (2), adapted to hold the device (1) raised to a certain distance on an horizontal flat surface(s), between which is standing up, also in horizontal position, a rod (3), where are inserted, with an axis-like turning movement, one or more rounded rotating parts (4) in which, in turn, are incorporated, fixed on its surface, magnets (5), so that the user, when passing the hand or fingers on the said parts (4) rotating them about the rod (3), receives the magnetic effect of the magnets (5).

Preferably, the magnets (5) are formed by circular parts that protrude above the rounded surface of the rotating parts (4) on which they are fixed, providing a massage on the user hands.

In an embodiment, the magnets (5) are joined integral to the surface of the rotating parts (4) in a fixed and stationary manner. However, in an alternative option of embodiment, one, several or all the magnets (5) are incorporated to the surface of the rotating parts (4) by means of a mobile union (5a), for example a spring, strip or similar system, that allows to exert pressure on them and always tends to return them to their initial position, as it is observed in the FIGS. 8-A and 8-B.

Preferably, the rotating parts (4) are spheric and possess a through hole (4a) to be inserted in the rod (3).

Preferably, the rod (3) incorporates three rotating parts (4).

Preferably, the support bases (2) are dismountable.

Preferably, the support bases (2) are fixed by means of snap-fit in cylindric bushings (6) in which the axis rod ends (3) are fixed.

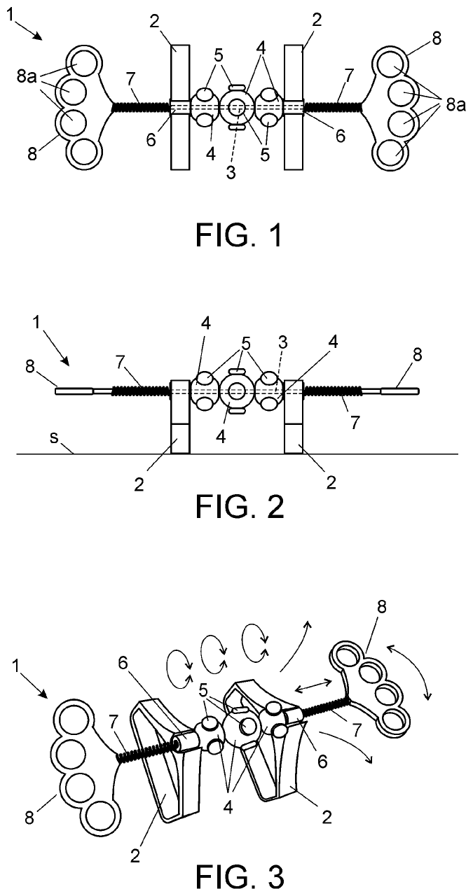

In a preferred embodiment, the bases (2), either dismountable or not, are formed, each, by a single piece, as it is shown in the example of the FIGS. 1 to 6.

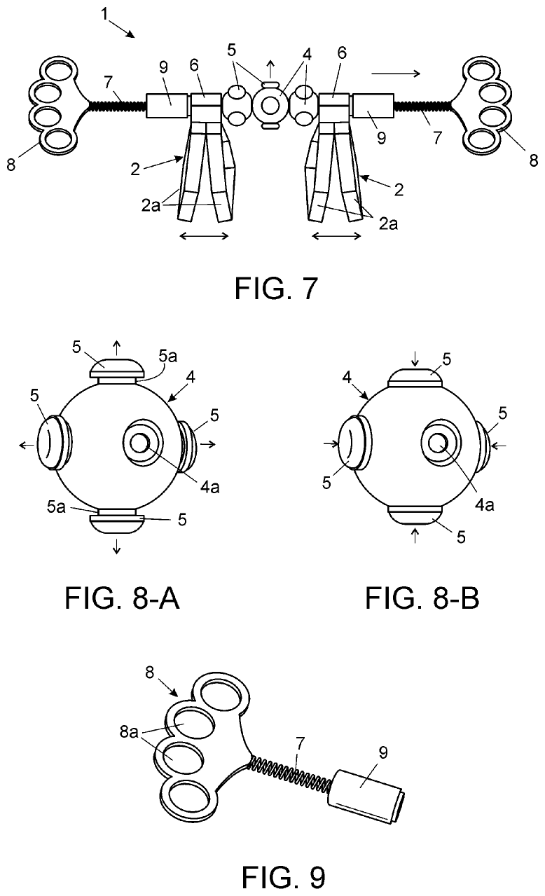

And, in an alternative embodiment, as shown in the FIG. 7, each of the two bases (2) of the device is formed by two other pieces (2a) hinged to each other by their top, where a bushing (6) links them to the rod (3), so that they can be more separated at their bottom, where they define the support of the assembly, to provide more support surface.

Preferably, on the lower face of the bases (2), as well if they are one piece as if they are of two pieces (2a), they incorporate a layer of anti-slip material, to prevent that the device moves when it is used.

In a preferred embodiment, the device (1) likewise comprises, coupled in each of the respective ends of the rod (3) that acts as axis for the rotating parts (4) a spring (7) or similar flexible threadlike element, at the distal end thereof, a side gripping part (8) is coupled, designed to be held with the hands, for which it is fitted with holes, concretely with four circular recesses (8a) adapted to place the fingers (index, middle, ring and little fingers) inserted in them, in order to apply movements in any direction to overcome the force of the springs (7) and with it, do rehabilitation exercises.

As it can be seen by the arrows of the FIG. 3, the said movements can be as well compressive, towards the central part of the device, or extensive in the opposite direction, as upwards and downwards or to one side or the other. Logically, the range of the said movements will depend on the length of the springs (7) and of their degree of flexibility, both things can vary depending on the preferences of the manufacturer, in each case.

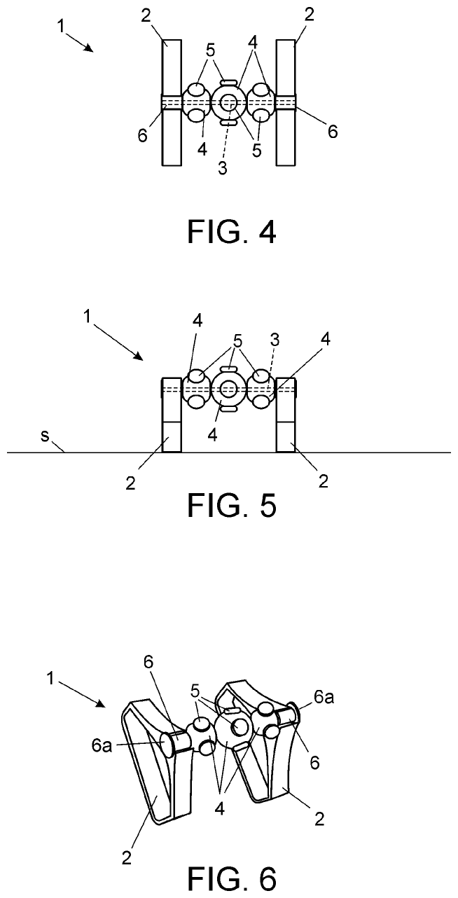

In the event that the device (1) does not comprise the springs (7) nor the gripping parts (8), preferably, as it is shown in the FIGS. 4, 5 and 6, the device incorporates two additional covers (6a) for covering the external holes of the bushings (6) of the bases (2) where the said springs (7) be, the central device remaining adapted to be used only for massage.

On the other hand, as shown in the example of the FIG. 7, in an option of embodiment, the springs (7) with the gripping parts (8) are dismountable, allowing their use independently (FIG. 9) as well as the use of the rest of the device (1) without them.

For this, preferably, the end of the spring (7) that is joined to the rod (3) possesses a stop (9) that, in addition to serve for its coupling in a withdrawable manner to the said rod (3), preferably, through a bushing (6) in which the bases (2) are fixed, for example by screwing or pressure, in turn also serves to use the gripping part (8) to do exercises with the hand, concretely inserting the fingers in the recesses (8a) and standing the said stop (9) on the palm of the hand for pressing, by closing the hand and overcome the force of the spring (7). If the said stop (9) did not exist, if the end of the spring (7) is directly standing, as it is pointed and metallic, it could cause injury to the user.

On the other hand, preferably, as well the gripping part (8) as the described stop (9) of the spring (7) end are made of silicone, plastic, rubber or other similar soft material, to prevent that it can hurt.

On the other hand, the sizes of the device are likewise variable, although, in the preferred embodiment, the distance between the two gripping parts (8) of the ends of the device and the rotating parts (4) of the rod (3) acting as axis is such that an adult user, when introducing the four fingers of his hands in the circular recesses (8a) thereof with each hand, can touch the surface with magnets (5) of the said rotating parts (4) with the thumbs.

Sufficiently described the nature of this invention, as well as the manner of implementing it, it is not deemed necessary to extend anymore its explanation in order that any person skilled in the art comprises its extend and the advantages arising from it.

1. A hand anti-stress and/or rehabilitation device, characterized in that it comprises two support bases (2) between which a metal rod (3) stands in which are inserted, with movement of rotation on itself like an axis, one or more rounded rotating parts (4) in which, in turn, magnets (5) are incorporated fixed on its surface.

2. The hand anti-stress and/or rehabilitation device according to claim 1, characterized in that the magnets (5) are constituted by circular parts that protrude above the rounded surface of the rotating parts (4) on which they are fixed.

3. The hand anti-stress and/or rehabilitation device according to claim 1, characterized in that the magnets (5) are incorporated integral with the surface of the rotating parts (4) in a fixed and stationary manner.

4. The hand anti-stress and/or rehabilitation device according to claim 1, characterized in that one, several or all the magnets (5) are incorporated to the surface of the rotating parts (4) by means of a mobile union (5a), that allows to exert pressure on them and always tends to return them to their initial position.

5. The hand anti-stress and/or rehabilitation device according to claim 1, characterized in that the rotating parts (4) are spheric.

6. The hand anti-stress and/or rehabilitation device according to claim 1, characterized in that the rod (3) incorporates three rotating parts (4).

7. The hand anti-stress and/or rehabilitation device according to claim 1, characterized in that the support bases (2) are dismountable.

8. The hand anti-stress and/or rehabilitation device according to claim 7, characterized in that the support bases (2) are fixed by means of snap-fit in cylindric bushings (6) in which the ends of the axis rod ends (3) are fixed.

9. The hand anti-stress and/or rehabilitation device according to claim 1, characterized in that the bases (2) are formed, each, by a single piece.

10. The hand anti-stress and/or rehabilitation device according to claim 1, characterized in that the support bases (2) are formed, each, by two other pieces (2a) hinged to each other by their top, so that they can be separated at their bottom, to provide more support surface.

11. The hand anti-stress and/or rehabilitation device according to claim 1, characterized in that it comprises, coupled in each of the respective ends of the rod (3) that acts as axis for the rotating parts (4) a spring (7) or similar flexible threadlike element, at the distal end thereof, a side gripping part (8) is coupled, fitted with holes, consisting in four circular recesses (8a) adapted to place the fingers in them.

12. The hand anti-stress and/or rehabilitation device according to claim 11, characterized in that the springs (7) with the gripping parts (8) are dismountable, allowing their use independently as well as the use of the rest of the device (1) without them.

13. The hand anti-stress and/or rehabilitation device according to claim 12, characterized in that the end of the spring (7) that is joined to the rod (3) possesses a stop (9) that serve for its coupling in a withdrawable and also to support the said stop (9) on the palm of the hand.

14. The hand anti-stress and/or rehabilitation device according to claim 13, characterized in that the gripping part (8) as the stop (9) of the spring (7) end are made of silicone, plastic, rubber or other similar soft material.

15. The hand anti-stress and/or rehabilitation device according to claim 11, characterized in that the distance between the two gripping parts (8) and the rotating parts (4) of the rod (3) is such that an adult user, when introducing his four fingers in the circular recesses (8a) thereof with each hand, can touch the surface with magnets (5) of the said rotating parts (4) with his thumbs.
